(19) United States
(12) Patent Application Publication
(10) Pub. No.: US 2019/0143042 A1
(43) Pub. Date: May 16, 2019

(54) EPINEPHRINE AUTO-INJECTING WATCH AND METHOD OF USE

(71) Applicant: Peter J. Hughes, II, Amarillo, TX (US)

(72) Inventor: Peter J. Hughes, II, Amarillo, TX (US)

(21) Appl. No.: 16/192,242

(22) Filed: Nov. 15, 2018

Related U.S. Application Data
(60) Provisional application No. 62/586,353, filed on Nov. 15, 2017.

(51) Int. Cl. A61M 5/20; A61M 5/32

(52) U.S. Cl. CPC A61M 5/2033; A61M 5/3202; A61M 5/31576; A61M 2005/206; A61M 5/326

(57) ABSTRACT

An epinephrine auto-injection device includes a wristband; an outer casing secured to the wristband, the outer casing having a time display thereon; an injection system housed within the outer casing, the injection system having a firing button engaged with a latch; a spring housed to exert force on the latch; a needle engaged with a plunger, the plunger being in communication with the latch; depressing the firing button releases the spring, thereby causing the spring to exert pressure on the latch; the latch exerts pressure on the plunger; and the plunger pushes the needle out of the outer casing and releases an epinephrine supply through the needle.

### BACKGROUND

### 1. Field of the Invention

The present invention relates generally to portable, personal medical devices, and more specifically, to a smart watch that is configured to inject epinephrine into the user for preventing or controlling allergic reactions.

### 2. Description of Related Art

Portable, personal medical devices are well known in the art and are effective means to monitor or control one's health. For example, FIG. 1 depicts a conventional epinephrine pen 101 having a safety cap 105 which is connected at the top of a body 103. At the bottom of the body 103 is the needle 107 which is surrounded by a protective cover 109. During use, the user removes the safety cap 105 while holding the body 103. The user then swings the body 103 and pushes the needle 107 into the thigh. After the injection is complete, the protective cover 109 surrounds and covers the needle 107.

One of the problems commonly associated with apparatus 101 is the device can be cumbersome or inconvenient to carry at all times. For example, the size and shape of the body 103 makes it difficult for a user to carry in a pocket or carry for an extended period of time. Due to this inconvenience, an individual may need epinephrine immediately and not have access to it or have to wait until some is located.

Also, in an emergency, it may difficult for the user to quickly remove the safety cap 105 and swing with enough force for the needle 107 to inject properly. Swinging with too much force may additionally cause other injuries.

Accordingly, although great strides have been made in the area of epinephrine pens, many shortcomings remain.

### DESCRIPTION OF THE DRAWINGS

The novel features believed characteristic of the embodiments of the present application are set forth in the appended claims. However, the embodiments themselves, as well as a preferred mode of use, and further objectives and advantages thereof, will best be understood by reference to the following detailed description when read in conjunction with the accompanying drawings, wherein:

FIG. 1 is a front view of a common epinephrine pen device;

FIG. 2 is a top view of an epinephrine auto-injecting watch device in accordance with a preferred embodiment of the present application;

FIGS. 3A and 3B are an internal views of the device of FIG. 2; and

FIG. 4 is a flowchart illustrating the method of use of the device of FIG. 2.

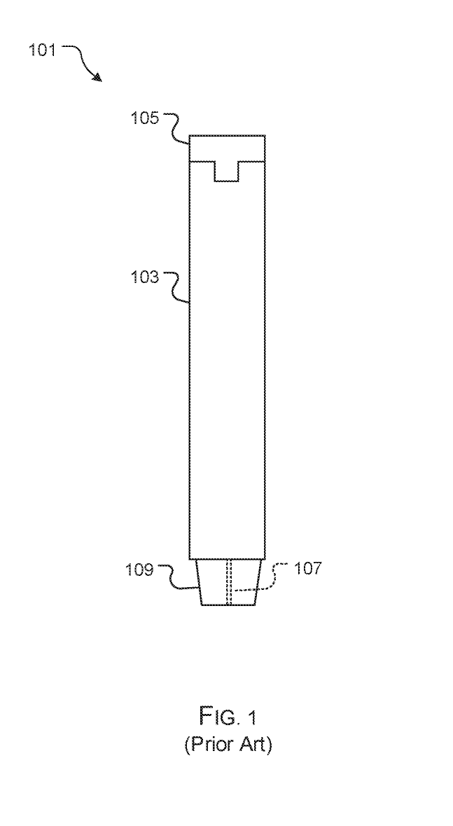

While the system and method of use of the present application is susceptible to various modifications and alternative forms, specific embodiments thereof have been shown by way of example in the drawings and are herein described in detail. It should be understood, however, that the description herein of specific embodiments is not intended to limit the invention to the particular embodiment disclosed, but on the contrary, the intention is to cover all modifications, equivalents, and alternatives falling within the spirit and scope of the present application as defined by the appended claims.

### DETAILED DESCRIPTION OF THE PREFERRED EMBODIMENT

Illustrative embodiments of the system and method of use of the present application are provided below. It will of course be appreciated that in the development of any actual embodiment, numerous implementation-specific decisions will be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The system and method of use in accordance with the present application overcomes one or more of the above-discussed problems commonly associated with conventional epinephrine pen devices. Specifically, the device can conveniently be worn on the wrist like a watch and can take the place of the user's watch. In addition, the simple release of the needle eliminates the need for “swinging” the device before injecting. These and other unique features of the system and method of use are discussed below and illustrated in the accompanying drawings.

The system and method of use will be understood, both as to its structure and operation, from the accompanying drawings, taken in conjunction with the accompanying description. Several embodiments of the system are presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless described otherwise.

The preferred embodiment herein described is not intended to be exhaustive or to limit the invention to the precise form disclosed. It is chosen and described to explain the principles of the invention and its application and practical use to enable others skilled in the art to follow its teachings.

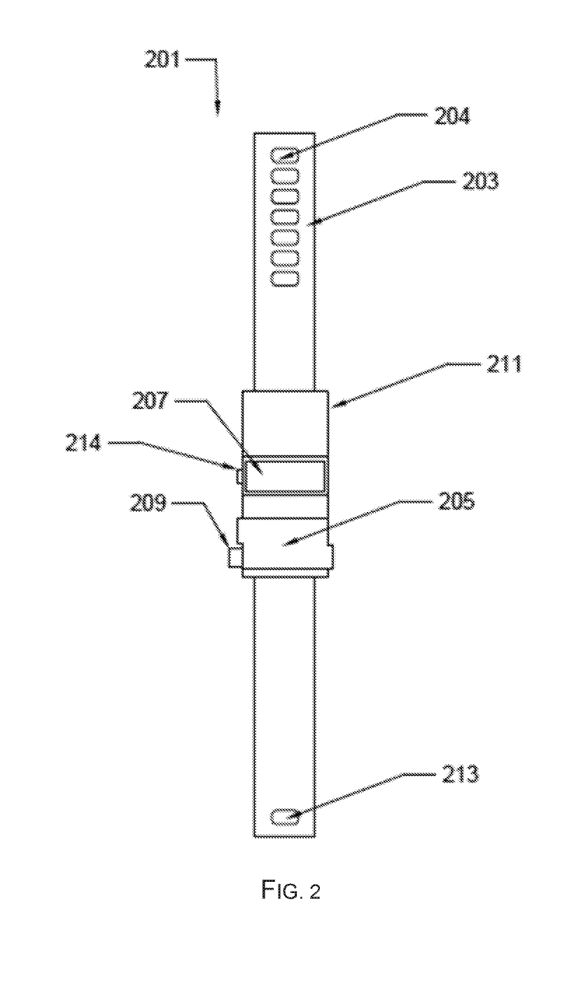

Referring now to the drawings wherein like reference characters identify corresponding or similar elements throughout the several views, FIG. 2 depicts a top view of an epinephrine auto-injecting watch device 201 in accordance with a preferred embodiment of the present application. It will be appreciated that device 201 overcomes one or more of the above-listed problems commonly associated with conventional epinephrine pen devices.

In the contemplated embodiment, device 201 includes a wristband 203 that connects to an outer casing 211 through means such as looping the band through connectors on the bottom of the casing 211, an adhesive, or any other means. The wristband 203 includes a clasp 213, can include one or more adjustment means 204, and is contemplated to be composed of a rubber, a silicone, a plastic, a leather or any other flexible, non-irritating material.

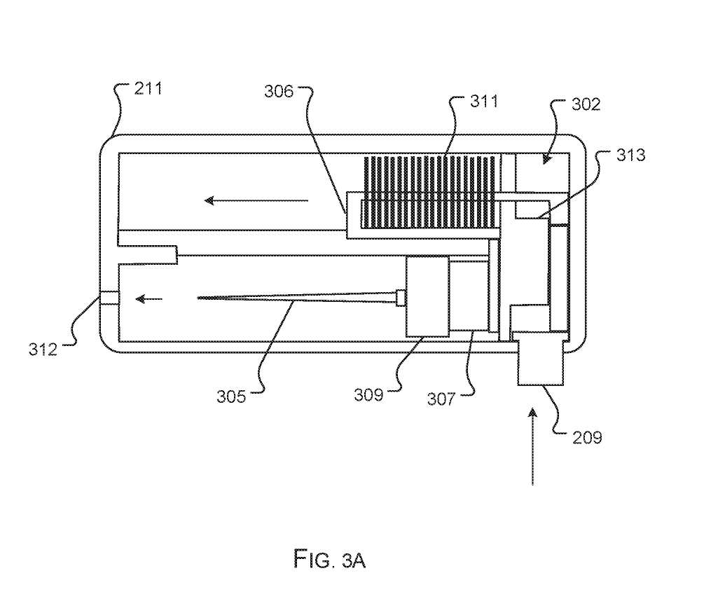

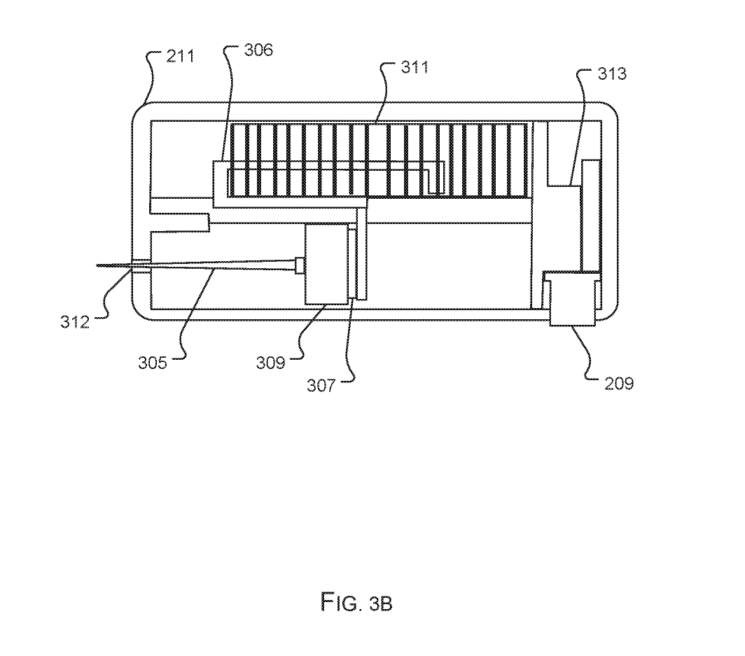

In the preferred embodiment, the casing 211 houses internal parts and elements configured to provide a means for injecting epinephrine, as shown in FIG. 3. The casing 211 also houses a time display 207, a screen wake button 214, a safety cap/pin 205, and a firing button 209. It should be understood that casing 211 will incorporate necessary elements for the time display to function, such as a power source, a time setting feature, a screen, and any other necessary element.

It should be appreciated that one of the unique features believed characteristic of the present application is the incorporation of a small epinephrine injection device into a wristband, thereby making the system convenient and easy to carry at all times. The ease and convenience is contemplated to reduce the risk of needing epinephrine and not having it, and to reduce the injection time.

Referring now to FIG. 3 of the preferred embodiment of an epinephrine auto-injecting watch, is an internal view of the casing 211, wherein a fire button 209 is in communication with a spring 311 loaded latch 306. It should be understood that safety pin/cap 205 (not shown in FIG. 3) prevents deployment of firing button 209 by engaging in internal area 302 of casing 211. The safety pin/cap 205 extends into area 302 to prevent the spring from firing when the button is deployed. When the firing button 209 is pressed, it applies pressure to latch 306 which in turn releases the latch from a barrier 313 allowing spring 311 to exert force on latch 306 and plunger 307 causing a forward motion. The plunger 307 then forces a needle 305 out of the device, through an opening 312 and an epinephrine supply 309 is released via the needle 305. The activation is shown in FIG. 3B for clarity.

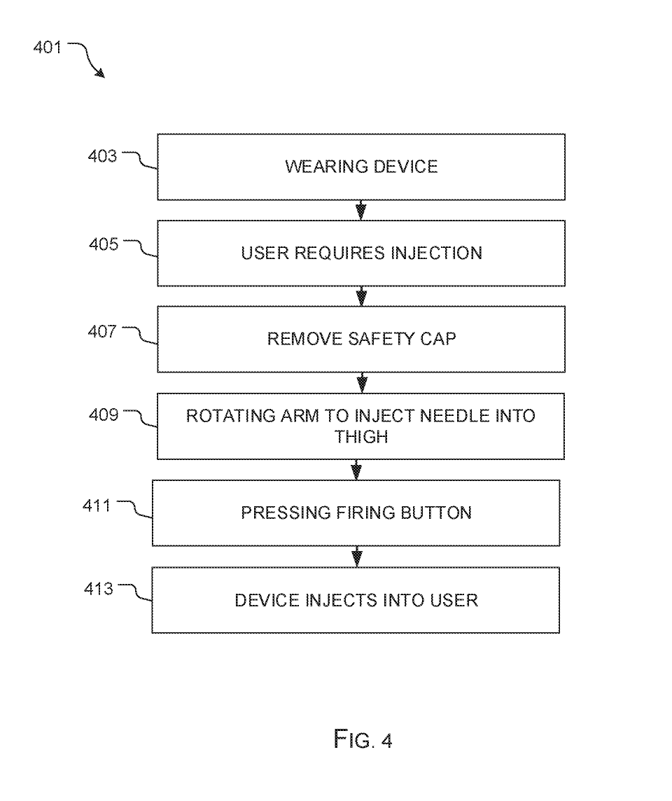

Referring now to FIG. 4 the preferred method of use 401 of device 201 is depicted. A user wears the device, preferably on a daily basis until the user needs the medication, as shown with boxes 403, 405. The user then quickly removes the safety pin/cap and rotates their arm to the desirable location to release the injection into their thigh, as shown with boxes 407, 409. The user then deploys the injection via the firing button, as shown with boxes 411, 413.

The particular embodiments disclosed above are illustrative only, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. Although the present embodiments are shown above, they are not limited to just these embodiments, but are amenable to various changes and modifications without departing from the spirit thereof.

What is claimed is:

1. An epinephrine auto-injection device, comprising:
a wristband;
an outer casing secured to the wristband, the outer casing having a time display thereon; and
an injection system housed within the outer casing, the injection system having:
a firing button engaged with a latch;
a spring housed to exert force on the latch; and
a needle engaged with a plunger, the plunger being in communication with the latch;
wherein depressing the firing button releases the spring, thereby causing the spring to exert pressure on the latch;
wherein the latch exerts pressure on the plunger; and
wherein the plunger pushes the needle out of the outer casing and releases an epinephrine supply through the needle.
